(19) United States
(12) Patent Application Publication
(10) Pub. No.: US 2022/0273569 A1
(43) Pub. Date: Sep. 1, 2022

(54) METHODS OF CREATING A SUBSTANCE WITH DIFFERENT FREEZING POINTS BY ENCAPSULATION

(71) Applicants: The General Hospital Corporation, Boston, MA (US); Brixton Biosciences, Inc., Cambridge, MA (US)

(72) Inventors: Richard Rox Anderson, Boston, MA (US); William Farinelli, Boston, MA (US); Lilit Garibyan, Newton, MA (US); Sameer Sabir, Arlington, MA (US); Charles Sidoti, Boston, MA (US); Mansoor M. Amiji, Attleboro, MA (US); Maie Taha, Giza (EG)

(73) Assignees: The General Hospital Corporation, Boston, MA (US); Brixton Biosciences, Inc., Cambridge, MA (US)

(21) Appl. No.: 17/629,206

(22) PCT Filed: Jul. 23, 2020
(86) PCT No.: PCT/US2020/043280

§ 371 Date: Jan. 21, 2022

Related U.S. Application Data
(60) Provisional application No. 62/878,108, filed on Jul. 24, 2019.

(51) Int. Cl. A61K 9/127; A61K 47/24; A61K 9/00; A61K 47/10

(52) U.S. Cl. CPC A61K 9/1277; A61K 47/24; A61K 9/0019; A61K 47/10

(57) ABSTRACT

The present disclosure relates to compositions and methods for manufacturing biomaterials that form flowable and injectable cold slurries. More particularly, the present disclosure relates to a composition containing a plurality of liposomes where the encapsulated internal liposomal media and external liposomal media have different freezing points.

### RELATED APPLICATIONS

The present application claims priority under 35 U.S.C. § 119(e) to U.S. Provisional Patent Application No. 62/878,108, filed Jul. 24, 2019, the contents of which are hereby incorporated by reference in their entirety.

### TECHNICAL FIELD

The present disclosure relates generally to compositions and methods for manufacturing biomaterials that form flowable and injectable cold slurries. More particularly, the present disclosure relates to a composition containing a plurality of liposomes where the encapsulated internal liposomal media and external liposomal media have different freezing points.

### BACKGROUND

Cold slurries (e.g., ice slurries) are known in the art as compositions that are made of sterile ice particles of water, varying amounts of excipients or additives such as freezing point depressants, and, optionally, one or more active pharmaceutical ingredients, as described in U.S. application Ser. No. 15/505,042 (“'042 Application”; Publication No. US2017/0274011), incorporated by reference in its entirety herein. The cold slurries can be delivered, preferably via injection, to a tissue of a subject, preferably a human patient, to cause selective or non-selective cryotherapy and/or cryolysis for prophylactic, therapeutic, or aesthetic purposes. Injectable cold slurries may be used for treatment of various disorders that require inhibition of nerve conduction. For example, U.S. application Ser. No. 15/505,039 (“'039 Application”; Publication No. US2017/0274078), incorporated by reference in its entirety herein, discloses the use of slurries to induce reversible degeneration of nerves (e.g., through Wallerian degeneration) by causing crystallization of lipids in the myelin sheath of nerves. The '039 Application also discloses using injectable cold slurries to treat various other disorders that require inhibition of somatic or autonomic nerves, including motor spasms, hypertension, hyperhidrosis, and urinary incontinence.

A method of preparing a cold slurry is shown in U.S. application Ser. No. 16/080,092 (“'092 Application”; Publication No. US2019/0053939). However, the '092 Application requires the point of care to manufacture the cold slurry by installing a medical ice slurry production system. This technique also requires the point of care take steps to maintain sterility of the cold slurry during manufacture and prior to administration.

There exists a need for compositions and methods that allow for simple transport, storage, and preparation of a flowable and injectable cold slurry at a clinical point of care without compromising the sterility of the slurry during preparation, without requiring manufacturing equipment to be available at the point of care, and without compromising the sterility of the biomaterial at the point of care. The present disclosure addresses this need by providing for improved compositions and methods that reduce the time required to provide a therapeutic substance, e.g., an injectable slurry, to a patient, that is easily shipped and stored.

### SUMMARY

The present disclosure provides compositions comprising a number of liposomes that separate an internal media from an external media where the internal media and external media have different freezing points, i.e., temperature at which the media freeze. The present disclosure further provides compositions that can be transformed into injectable slurries at the point of care by being placed into a standard freezer due to the different freezing points of the compositions.

In one aspect, disclosed herein is a composition comprising water; at least one liposome; and at least one excipient; wherein the liposome is configured to encapsulate a first volume of the composition, wherein the excipient is configured to be confined to a second volume of the composition external to the liposome and is configured to be separated from the encapsulated first volume, wherein a first freezing point of the encapsulated first volume is greater than a second freezing point of the second volume.

In some embodiments, the liposome is comprised of a lipid selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-α-phosphatidylcholine (PC), phosphatidylethanolamine, (PE), phosphatidylserine (PS), phosphatidylglycerol (PG), and a combination thereof. In some embodiments, the lipid is L-α-phosphatidylcholine (PC).

In some embodiments, the excipient is selected from the group consisting of a salt, an ion, Lactated Ringer's solution, a sugar, a biocompatible surfactant, a polyol, and a combination thereof. In some embodiments, the excipient is a polyol. In some embodiments, the polyol is polyethylene glycol 1000 (PEG 1000).

In some embodiments, the composition further includes a second excipient in both the first and second volumes. In some embodiments, the second excipient is saline or phosphate-buffered saline (PBS).

In some embodiments, the encapsulated first volume is between about 20% and about 50% of a total volume of the composition. In some embodiments, the encapsulated first volume is between about 30% and about 40% of a total volume of the composition. In some embodiments, the encapsulated first volume is between about 40% and about 50% of a total volume of the composition. In some embodiments, the encapsulated first volume is between about 35% and about 40% of a total volume of the composition. In some embodiments, the encapsulated first volume is between about 40% and about 45% of a total volume of the composition. In some embodiments, the encapsulated first volume is about 38% of the total volume of the composition. In some embodiments, the encapsulated first volume is about 43% of the total volume of the composition.

In some embodiments, the first freezing point of the encapsulated first volume is between about −2° C. and about 0° C. In some embodiments, the second freezing point of the second volume is between about −20° C. and about −10° C. In some embodiments, an average freezing point of a total volume of the composition comprising the first volume, the second volume, and the liposome is between about −10° C. and about −5° C.

In some embodiments, the encapsulated first volume is configured to form a plurality of ice particles when the composition is cooled to a predetermined temperature. In some embodiments, the ice particles comprise between about 30% by weight and about 50% by weight of the total weight of the composition. In some embodiments, the predetermined temperature is between about −20° C. and about −5° C. In some embodiments, the predetermined temperature is about −20° C. In some embodiments, the predetermined temperature is about −5° C.

In another aspect, disclosed herein is a method of preparing a composition for administration to a patient at a clinical point of care, the method comprising preparing a composition comprising a plurality of liposomes, wherein an aqueous medium fills an intraliposomal volume and an extraliposomal volume; adding at least one excipient to the extraliposomal volume, wherein the at least one excipient reduces a first freezing point of the extraliposomal volume below a second freezing point of the intraliposomal volume; and cooling the composition to a predetermined temperature such that a cold slurry is formed having a plurality of ice particles within the intraliposomal volume.

In some embodiments, the liposomes are comprised of a lipid selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-α-phosphatidylcholine (PC), phosphatidylethanolamine, (PE), phosphatidylserine (PS), phosphatidylglycerol (PG), and a combination thereof. In some embodiments, the lipid is L-α-phosphatidylcholine (PC).

In some embodiments, the excipient is selected from the group consisting of a salt, an ion, Lactated Ringer's solution, a sugar, a biocompatible surfactant, a polyol, and a combination thereof. In some embodiments, the excipient is a polyol. In some embodiments, the polyol is polyethylene glycol 1000 (PEG 1000).

In some embodiments, the aqueous medium is comprised of water, saline, or phosphate-buffered saline (PBS).

In some embodiments, the intraliposomal volume is between about 20% and about 50% of a total volume of the composition. In some embodiments, the intraliposomal volume is between about 30% and about 40% of a total volume of the composition. In some embodiments, the intraliposomal volume is between about 40% and about 50% of a total volume of the composition. In some embodiments, the intraliposomal volume is between about 35% and about 40% of a total volume of the composition. In some embodiments, the intraliposomal volume is between about 40% and about 45% of a total volume of the composition. In some embodiments, the intraliposomal volume is about 38% of the total volume of the composition. In some embodiments, the intraliposomal volume is about 43%.

In some embodiments, the first freezing point of the extraliposomal volume is between about −20° C. and about −10° C. In some embodiments, the second freezing point of the intraliposomal volume between about −2° C. and about 0° C. In some embodiments, an average freezing point of a total volume of the composition comprising the first volume, the second volume, and the liposomes, is between about −10° C. and about −5° C.

In some embodiments, the ice particles comprise between about 30% by weight and about 50% by weight of the biomaterial.

In some embodiments, the predetermined temperature is between about −20° C. and −5° C. In some embodiments, the predetermined temperature is about −20° C. In some embodiments, the predetermined temperature is about −5° C.

In some embodiments, a bilayer configuration of the liposomes is chosen from the group consisting of unilamellar vesicles, multilamellar vesicles, oligolamellar vesicles, multivesicular vesicles, and a combination thereof.

In some embodiments, the liposomes have an average diameter of between about 0.1 μm and about 2 μm.

In another aspect, disclosed herein is a method of making a flowable and injectable encapsulated ice solution, the method comprising providing a plurality of biodegradable liposomes configured to form vesicles selected from the group consisting of multilamellar, oligolamellar, multivesicular, giant unilamellar, large unilamellar, small unilamellar, or a combination thereof; entrapping water within at least two of the plurality of liposomes to generate liposomes filled with a first volume comprising water; adding an excipient to an extraliposomal second volume separated from the first volume, wherein the excipient alters the freezing point of the second volume relative to the first volume; freezing the plurality of filled liposomes to generate a plurality of ice particles inside the filled liposomes; and controlling an average diameter of each of the plurality of ice particles to a predetermined size.

In some embodiments, the first volume is between about 20% and about 50% of a total volume of the composition. In some embodiments, the first volume is between about 20% and about 50% of a total volume of the composition. In some embodiments, the first volume is between about 30% and about 40% of a total volume of the composition. In some embodiments, the first volume is between about 40% and about 50% of a total volume of the composition. In some embodiments, the first volume is between about 35% and about 40% of a total volume of the composition. In some embodiments, the first volume is between about 40% and about 45% of a total volume of the composition. In some embodiments, the first volume is about 38% of the total volume of the composition. In some embodiments, the first volume is about 43% of the total volume of the composition.

In some embodiments, the excipient is PEG 1000.

### BRIEF DESCRIPTION OF THE DRAWINGS

The following figures depict illustrative embodiments of the present disclosure.

FIG. 1 is a diagram of a composition containing liposomes with differential freezing points across the intraliposomal and extraliposomal media.

FIG. 2 depicts a freezing point depression graph for water and a solution containing 47% PEG 1000 volume by volume (“v/v”).

FIG. 3 is a graph of solid to liquid phase transitions of cold slurries having a crystallization set point of −6.5° C.

### DETAILED DESCRIPTION

The present disclosure is directed to compositions and methods of preparing an injectable biomaterial, such as a sterile cold slurry. The biomaterial preferably contains a suspended material, preferably liposomes, that separate an internal media from an external media that have different freezing points. Due to the different freezing points of the internal and external media, the liposomes are preferably able to encapsulate internal media that will freeze while the external media remains in a liquid state at a given temperature, e.g., 0° C. In a preferred embodiment, the biomaterial forms a flowable and injectable slurry that contains a plurality of ice particles. The ice particles are preferably held within a plurality of liposomes and are kept separate from a liquid solution by the liposomal barrier. The internal media is preferably pure water and the external media is preferably a solution including water and non-active excipient materials. In other embodiments, the slurry further comprises a known active pharmaceutical compound.

The present disclosure is directed to a flowable and injectable ice slurry containing ice particles that have a precise particle size distribution, and the slurry is biodegradable, biocompatible and able to be stored for long durations (e.g., two or more years). In some embodiments, pure water or saline is encapsulated within biocompatible and biodegradable material, such as a liposome, which can separate an aqueous phase solution from a solid phase material when the composition is placed into a standard freezer, for example a freezer set at about −20° C., to create flowable and injectable encapsulated ice particles. The different freezing points are created by adding freeze point depressant material to the extraliposomal media. Encapsulating water allows for the control of the size and shape of the ice particles when the biomaterial is exposed to freezing temperature conditions. An example of material that can be used to encapsulate water/ice is liposomes. Liposomes have been widely used in medicine for delivery of active molecules and drugs to a target tissue. However, the present disclosure is directed to using liposomes to encapsulate ice particles creating a cold slurry composition as a therapeutic biomaterial.

In some embodiments, the biomaterial is a cold slurry (e.g., ice slurry) that can be delivered via injection directly to tissue of a human patient or a subject for prophylactic, therapeutic, or aesthetic purposes. The injectable slurry can be used for selective or non-selective cryotherapy or cryolysis.

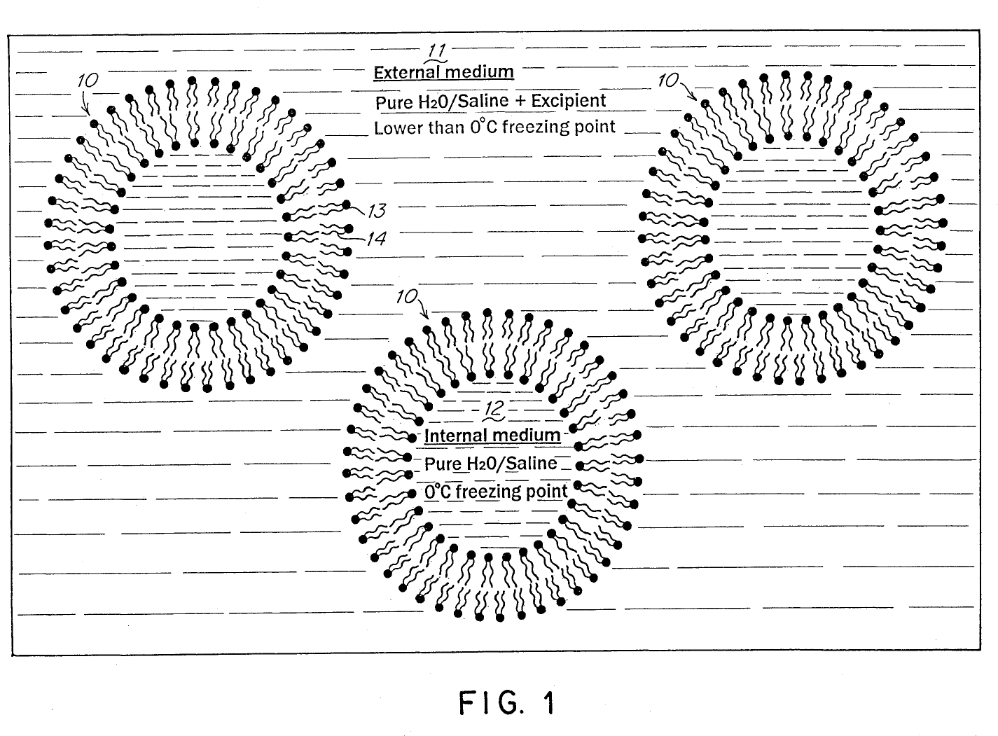

In some embodiments, liposomes are used to create a solution or mixture with differential freezing points for the purpose of creating a flowable cold slurry. Referring to FIG. 1, a diagram of a biomaterial shows orthographic views of liposomes having an internal medium composed of water (or saline) with a freezing point of 0°, and an external medium in which the liposomes are suspended which is composed of water (or saline) and at least one excipient (e.g., polyethylene glycol 1000, “PEG 1000”) with a freezing point that is lower than 0° C. In some embodiments, the freezing point of the intraliposomal medium is less than about −2° C., between about −2° C. and about 0° C., between about 0° C. and about 2° C., or greater than about 2° C. In some embodiments, the freezing point of the extraliposomal medium is less than about −15° C., between about −15° C. and about −10° C., between about −10° C. and about −5° C., or greater than about −5° C. Given the different freezing points across the internal and external media, when the biomaterial is subjected to cooling at specific temperatures, ice particles are formed in the intraliposomal medium, while leaving the extraliposomal medium in an aqueous state, creating a flowable and injectable cold slurry composition. In some embodiments, the biomaterial can also be allowed to have ice particles that partially melt before administering the biomaterial to a patient to create an injectable and flowable slurry.

Liposomes are sphere-shaped vesicles that can be created from nontoxic lipids/phospholipids. As shown in FIG. 1, phospholipids have a hydrophilic head group 13 and two long hydrophobic tails 14, giving them the propensity to self-assemble into vesicular bilayers when suspended in water. In some embodiments, the liposomes are synthesized from commonly used lipids/phospholipids known in the art such as 1,2-distearoyl-sn-glycero-3-phosphocholine (DSPC), egg sphingomyelin (DPSM), dipalmitoylphosphatidyl (DPPC), dicethylphosphate (DCP), L-α-phosphatidylcholine (e.g., egg PC or soy PC), phosphatidylethanolamine, (e.g., egg PE or soy PE), phosphatidylserine (PS) and phosphatidylglycerol (PG), or any combination thereof. Various phospholipids can be selected to create liposomes of desired levels of fluidity and permeability. In preferred embodiments, the liposome composition includes cholesterol to improve the stability of the bilayers and reduce lipid aggregates. Liposomes may additionally be synthesized (or coated) with polymers such as poly(lactic-co-glycolic acid) (PLGA), or polyethylene glycol (PEG) to improve stability. In some embodiments, the liposomes additionally comprise one or more surfactants (e.g., sodium cholate). In preferred embodiments, the liposomes are made entirely of biodegradable and non-immunogenic components.

In some embodiments, the liposomes will consist of one or more phospholipid bilayers. In some embodiments, liposomes are unilamellar vesicles, i.e., vesicles composed of a single lipid bilayer, (such as those depicted in FIG. 1) including giant unilamellar vesicles (GUV; >1 μm (diameter of the vesicle)), large unilamellar vesicles (LUV; >0.1 μm) and small unilamellar vesicles (SUV; <0.1 μm). In some embodiments, the liposomes are multilamellar/oligolamellar vesicles (MLV/OLV) composed of multiple lipid bilayers organized in concentric phospholipid spheres. In some embodiments, the liposomes are multivesicular vesicles (MVV) composed of multiple non-concentric vesicles encapsulated within a single bilayer. In some embodiments, the phospholipid charge of the liposome is neutral, anionic, or cationic. The lipid composition, lipid chain length and saturation, size, method preparation and charge of the vesicles can all be modulated to change liposome properties. In some embodiments, the liposomes are synthesized with short and unsaturated chains of phospholipids to allow separation of the aqueous phase outside the bilayer from the solid ice inside without freezing or deforming of the liposomes.

Any method known in the art may be used to prepare the liposomes as disclosed herein. For example, liposomes according to the present disclosure may be made according to the methods disclosed in Dua J. S., et al., Liposome: methods of preparation and applications, 3 Int. J. Pharm. Stud. Res. 14-20 (April 2012), and incorporated by reference in its entirety herein. Such methods include mechanical dispersion methods (e.g., lipid film hydration, sonication, freeze-drying, freeze-thaw, French pressure cell, micro-emulsification), solvent dispersion methods (e.g., ethanol injection, reverse phase evaporation, double emulsion), and detergent removal methods (e.g., dialysis, dilution, column chromatography). In some embodiments, the sonication of the mechanical dispersion method is used to create small liposomal vesicles of given diameters as disclosed further herein. The liposomes are created in a medium that will allow entrapping of pure water, saline, or phosphate buffered saline (PBS) (i.e., the intraliposomal medium). These liposomes may be freeze-dried and later rehydrated in another medium (i.e., the extraliposomal medium). In some embodiments, the extraliposomal medium is composed of a solvent (e.g., pure water, saline, or phosphate buffered saline) and at least one excipient (e.g., PEG 1000) that will serve as a freezing point depressant (see FIG. 1).

The present disclosure is also directed to making flowable and injectable encapsulated ice solutions that can be made at a central location and shipped to a point of care at room temperature (e.g., about 19° C.) and quickly converted into ice slurries at the point of care by simply reducing the temperature of the biomaterial using a standard freezer. This allows the point of care to not manufacture the biomaterial or be concerned with maintaining sterility of the biomaterial. The aqueous biomaterial containing the liposomes may be placed in a standard freezer at the clinical point of care set to a temperature of colder than about −25° C., between about −25° C. and about −20° C., between about −20° C. and about −15° C., between about −15° C. and about −10° C., between −10° C. and about −5° C., between about −5° C. and about 0° C., and warmer than about 0° C. In some embodiments, the biomaterial is placed into the freezer for a predetermined amount of time such that the temperature of the biomaterial drops to a desired level for forming a cold slurry with a given percentage of ice particles.

In some embodiments, the final liposomal composition (with an internal and external medium and lipids) is subjected to sterilization and remains sterilized from the point of manufacture and loading into a delivery vessel (e.g., bag or syringe) to the point of administration. In some embodiments, the internal and/or external liposomal media are sterilized during liposomal preparation and remain sterilized throughout the entire manufacturing, transportation, and storage process. In some embodiments, the liposomal composition is sterilized at the point of care using any sterilization methods known in the art (e.g., using heat, irradiation, high pressure, etc.). In some embodiments, the liposomal composition is sterilized while inside of a vessel (e.g., bag or syringe).

In some embodiments, the biomaterial is turned into a cold slurry through snap freezing. In such embodiments, ice particles are created within liposomes by changing of pressure. When pure water freezes, it expands. Starting with specific shapes or sizes of encapsulated water, temperature that is reduced below 0° C. under high pressure cannot freeze until that pressure is released, allowing the water to expand and therefore cause snap freezing of the intraliposomal volume. With snap freezing, a thermal gradient is not required.

The disclosed liposome technology allows the creation of fixed size liposomes for various applications. In some embodiments, intense sonication during preparation of the liposomes is used to limit the size of phospholipids to ensure that they will be injectable. Size can also be controlled by creating minimum lamellar size that is energetically favorable and prevent diffusion out of the intraliposomal volume. The free energy barrier of such minimally sized liposomes will trap water in a setting of higher osmolality outside of the liposomal vesicles. The disclosed methods allow for a cold slurry solution with very precise particle sizes with a wide range from about 0.02 μm to about 100 μm in diameter. In preferred embodiments, the average diameter of liposomes in the composition is less than about 0.1 μm, between about 0.1 μm and about 0.5 μm, between about 0.5 μm and about 1 μm, between about 1 μm and about 1.5 μm, between about 1.5 μm and about 2 μm, or greater than about 2 μm. In some embodiments, the average diameter of liposomes in the composition is between about 0.2 μm and about 0.4 μm, or between about 1.1 μm and about 1.3 μm.

The distribution of liposomal sizes is measured using standard techniques known in the art such as using electron microscopy, dynamic light scattering (DLS), atomic force microscopy (AFM), size exclusion chromatography (SEC), etc. In some embodiments, the sizes of the ice particles will be controlled to allow for flowability through a vessel of various sizes (e.g., needle gauge size of between about 7 and about 43) as described in the '042 Application, incorporated by reference in its entirety herein. In some embodiments, the average diameter is measured by dynamic light scattering (DLS).

In some embodiments, the average diameter is a mean diameter.

In some embodiments, one or more excipients are included in the slurry. An excipient is any substance, not itself a therapeutic agent, used as a diluent, adjuvant, and/or vehicle for delivery of a therapeutic agent to a subject or patient, and/or a substance added to a composition to improve its handling, stability, or storage properties. In order to create a biomaterial with differential freezing points across the intraliposomal and extraliposomal media, one or more freezing point depressants can be added as excipients to the extraliposomal solution to lower the freezing point of the extraliposomal solution (e.g., below about 0° C.). After the liposomes are prepared and suspended in an aqueous medium, the excipient is added to the external medium. Depressing the freezing point of the extraliposomal medium allows the final slurry mixture to maintain flowability and remain injectable while still containing an effective percentage of ice particles. Suitable freezing point depressants include salts (e.g., sodium chloride, betadex sulfobutyl ether sodium), ions, Lactated Ringer's solution, sugars (e.g., glucose, sorbitol, mannitol, hetastarch, sucrose, (2-Hydroxypropyl)-β-cyclodextrin, or a combination thereof), biocompatible surfactants such as glycerol (also known as glycerin or glycerine), other polyols (e.g., polyvinyl alcohol, polyethylene glycol 300, polyethylene glycol 400, polyethylene glycol 1000, propylene glycol), other sugar alcohols, or urea, and the like. Other exemplary freezing point depressants are disclosed in the '042 Application and are incorporated by reference in their entirety herein.

Preferably, the freeze point depressant added to the extraliposomal medium is polyethylene glycol 1000 (PEG 1000). PEG 1000 is particularly suited for the current disclosure due to its large molecular weight/size (i.e., about 1000 kDa) which prevents it from permeating the lipid membranes of the liposomes and entering the intraliposomal medium, which would degrade the freezing point differential created across the liposome. Other suitable freeze point depressants include any excipients which reduce the freezing point of the extraliposomal medium without permeating the membranes or degrading the freezing point differential.

The concentrations of freezing point depressants will determine the ice particle percentage of the slurry and its flowability and injectability. In some embodiments, the freezing point depressant (e.g. PEG 1000) makes up between about 10% v/v and about 70% v/v of the extraliposomal medium. In some embodiments, the freezing point depressant makes up less than about 30% v/v, between about 30% v/v and about 40% v/v, between about 40% and about 50% v/v, between about 50% and about 60% v/v, or more than about 60% v/v of the extraliposomal medium.

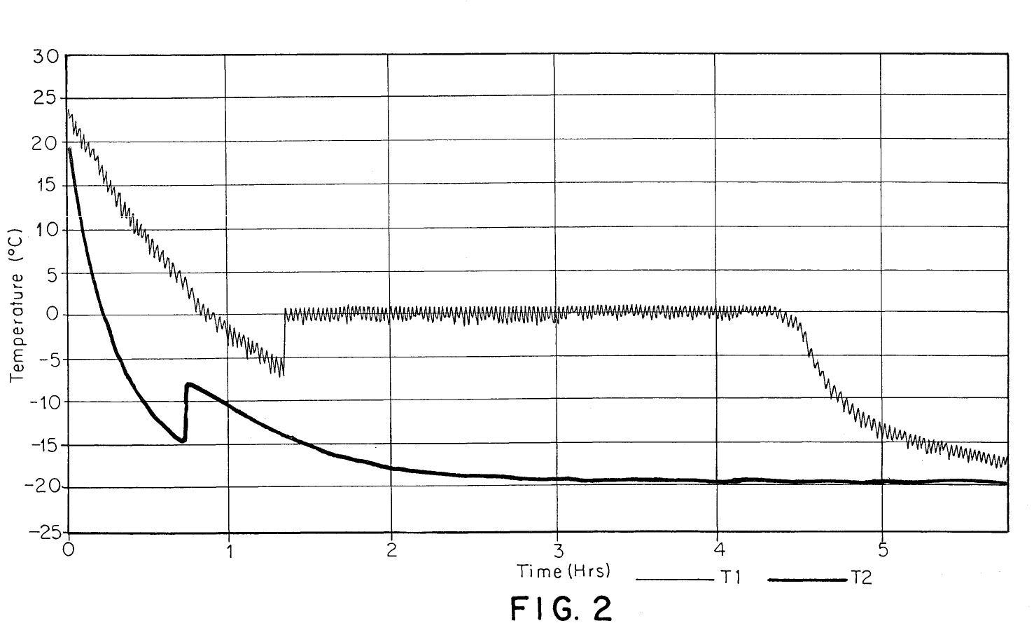

Referring to FIG. 2, a freezing point depression graph is shown for pure water T1 and a mixture of water and 47% v/v PEG 1000 T2. In this graph, all the substances were placed in a freezer having a constant temperature of −20° C. The temperature was measured using a thermometer placed in each substance/slurry. The graph shows that a mixture of water and PEG 1000 will have a different freezing point than that of pure water, which means the solution can be cooled to below 0° C. and become only partially crystallized. The graph shows that cooling causes pure water T1 to crystallize at an equilibrium freezing point of 0° C. This is indicated by the period of time where the pure water T1 remains at a temperature of about 0° C., from about 1.3 hours to about 4.4 hours, which begins immediately after pure water T1 passes a supercooling point at about −6° C. Having an equilibrium window of crystallization (i.e., the “flat line” portion of pure water T1 in FIG. 2) is typical for a pure solvent. For the 47% PEG 1000 solution T2, cooling causes the solution to begin crystallizing at an initial freezing point of about −6.5° C. after about just under 1 hour, and the crystallization continues as the temperature of the solution drops further to about −19° C. after about 2.5 hours. The initial crystallization occurs immediately after 47% PEG 1000 solution T2 passes a supercooling point at about −15° C., shown after about just under 1 hour. In some embodiments, having a descending temperature window of crystallization for the 47% PEG 1000 solution T2 is typical for a solution (i.e., impure mixture).

In some embodiments, the final product to be administered via injection to a human patient or a subject (such as a human who is not a patient or a non-human animal) is a cold slurry comprised of sterile ice particles of water, lipids forming liposomes, and varying amounts of excipients or additives such as freezing point depressants (e.g., PEG 1000).

In some embodiments, the ice particles are generally restricted to the intraliposomal medium. In some embodiments, the total volume of the intraliposomal medium is crystallized either entirely or partially.

In some embodiments, the percentage of ice particles in the total volume of the cold slurry composition constitutes less than about 10% by weight of the slurry, between about 10% by weight and about 20% by weight, between about 20% by weight and about 30% by weight, between about 30% by weight and about 40% by weight, between about 40% by weight and about 60% by weight, more than about 60% by weight, and the like.

In some embodiments, the sizes of the ice particles are controlled to allow for flowability through a vessel of various sizes (e.g. needle gauge size of between about 7 and about 43) as described in the '042 Application and incorporated by reference herein. In some embodiments, the biomaterial is first cooled to a specific temperature (as disclosed previously herein) and is further subject to thawing to achieve the desired percentage of ice particles.

The percentage of ice particles in the slurry composition can be partially controlled through the encapsulated volume in the liposomal composition. The greater the encapsulated volume, the greater the final percentage of ice particles when the composition is placed in a freezer. The encapsulated volume is the percentage of the total volume of the composition that is located within the intraliposomal medium (e.g., 40% encapsulated volume means that 40% of the composition is made up of the intraliposomal medium and 60% of the composition is made up of the lipids, excipients, and the extraliposomal medium). In some embodiments, the encapsulated volume of the biomaterial is less than about 20%, between about 20% and about 30%, between about 30% and about 40%, between about 40% and about 50%, or greater than about 50%. In some embodiments, the encapsulated volume is about 38%. In alternative embodiments, the encapsulated volume is about 43%. In some embodiments, the desired encapsulated volume is achieving using multiple filtrations of the composition to reduce the volume of the extraliposomal medium while concentrating the liposomes. The encapsulated volume can be estimated during preparation of the biomaterial using methods known in the art, including the method described in Oku, N, et al., A simple procedure for the determination of the trapped volume of liposomes, 691 Biochim. Biophys. Acta 332-340 (1982), incorporated by reference in its entirety herein. Briefly, Oku described preparing liposomes in a solution containing the fluorescent dye calcein. Once the liposomes are formed, cobalt cation is added to the external medium which acts to quench the fluorescence of calcein only in the external medium, and therefore the entrapping volume is the percentage of fluorescence that remains after the quenching occurs. Other standard methods known in the art to determine entrapping volume may also be used with the present disclosure.

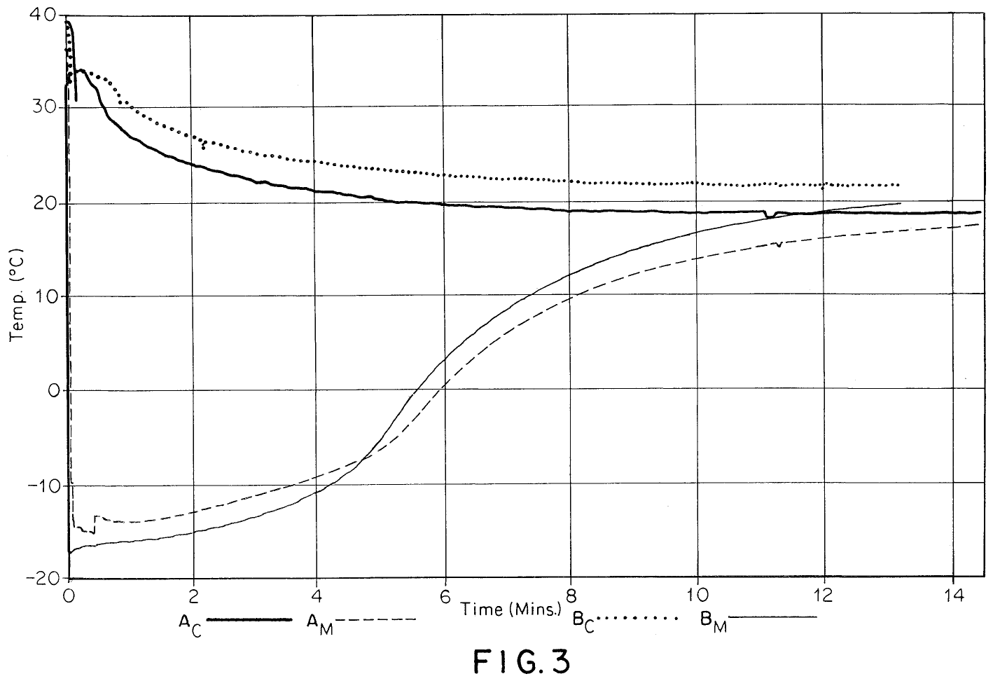

Referring to FIG. 3, two different slurry compositions (batches) are characterized with respect to their temperature profiles. The temperature traces show two separately created slurry batches having the same composition: 47% v/v PEG 1000 in the extraliposomal medium and 38% liposomal entrapping volume, with a measured freezing point of −6.5° C. The two slurry batches were placed into a copper plate that is heated to 40° C. and has thermocouple wires that measure changes in temperature of the slurry and the copper plate over time. The plotted data shows temperature change over time for two different slurry batches that were both cooled to −18° C. in a freezer immediately before being placed onto the heated copper plate. The temperatures are measured at two different positions for each slurry: embedded inside of the copper plate (traces A_{C} and B_{C}) and in the middle of the copper plate exposed to the outside of the plate (traces A_{M} and B_{M}). When a slurry batch is first introduced into the copper plate, the thermocouple wire embedded inside the plate (traces A_{C} and B_{C}) initially measures the warm temperature of the heated plate (about 38° C. for both traces A_{C} and B_{C} at timepoint 0) and then reaches an equilibrium at a lower temperature due to the cooling effect of the introduced slurry (19° C. for trace A_{C} at around 5 minutes and 24° C. for trace B_{C} at around 6 minutes). On the other hand, for the thermocouple wire located in the middle of the plate, when a slurry is first introduced into the copper plate it immediately contacts the thermocouple wire since that wire is exposed. This causes an initially negative temperature reading in the middle position due to the crystallized slurry contacting the wire (−15° C. for trace A_{M} and −17° C. for trace B_{M} at timepoint 0) followed by an equilibrium at a warmer temperature as the slurry melts on the heated plate (16° C. for trace A_{M} at around 12 minutes and 19° C. for trace B_{M} at around 8 minutes). The thermocouple wire exposed to the slurry (traces A_{M} and B_{M}) can be used to detect phase transitions during which the crystallized slurry begins to melt. The graph shows that both slurry compositions reach their phase transition at similar timepoints (at around 5 minutes for both traces A_{M}, and B_{M}). The graph also shows that the two slurry batches reach equilibrium (as measured by the two thermocouple wire positions) in a similar time frame and at similar temperatures of between about 17° C. and about 24° C. depending on the location of the thermocouple (middle/bottom). FIG. 3 therefore demonstrates that batch to batch consistency exists across slurries having the same composition.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising,” “including,” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A composition comprising:
water;
at least one liposome; and
at least one excipient;
wherein the liposome is configured to encapsulate a first volume of the composition,
wherein the excipient is configured to be confined to a second volume of the composition external to the liposome and is configured to be separated from the encapsulated first volume, and
wherein a first freezing point of the encapsulated first volume is greater than a second freezing point of the second volume.

2. The composition of claim 1, wherein the liposome is comprised of a lipid selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-α-phosphatidylcholine (PC), phosphatidylethanolamine, (PE), phosphatidylserine (PS), phosphatidylglycerol (PG), and a combination thereof.

3. The composition of claim 2, wherein the lipid is L-α-phosphatidylcholine (PC).

4. The composition of any preceding claim, wherein the excipient is selected from the group consisting of a salt, an ion, Lactated Ringer's solution, a sugar, a biocompatible surfactant, a polyol, and a combination thereof.

5. The composition of any preceding claim, wherein the excipient is a polyol.

6. The composition of claim 5, wherein the polyol is polyethylene glycol 1000 (PEG 1000).

7. The composition of any preceding claim, wherein the composition further includes a second excipient in both the first and second volumes.

8. The composition of claim 7, wherein the second excipient is saline or phosphate-buffered saline (PBS).

9. The composition of any preceding claim, wherein the encapsulated first volume is between about 20% and 50% of a total volume of the composition.

10. The composition of claim 9, wherein the encapsulated first volume is about 38% of the total volume of the composition.

11. The composition of claim 9, wherein the encapsulated first volume is about 43% of the total volume of the composition.

12. The composition of any preceding claim, wherein the first freezing point of the encapsulated first volume is between about −2° C. and about 0° C.

13. The composition of any preceding claim, wherein the second freezing point of the second volume is between about −20° C. and about −10° C.

14. The composition of any preceding claim, wherein an average freezing point of a total volume of the composition comprising the first volume, the second volume, and the liposome is between about −10° C. and about −5° C.

15. The composition of any preceding claim, wherein the encapsulated first volume is configured to form a plurality of ice particles when the composition is cooled to a predetermined temperature.

16. The composition of claim 15, wherein the ice particles comprise between about 30% by weight and about 50% by weight of the total weight of the composition.

17. The composition of any one of claim 15 or 16, wherein the predetermined temperature is between about −20° C. and −5° C.

18. A method of preparing a composition for administration to a patient at a clinical point of care, the method comprising:
preparing a composition having a plurality of liposomes, wherein an aqueous medium fills an intraliposomal volume and an extraliposomal volume;
adding at least one excipient to the extraliposomal volume, wherein the at least one excipient reduces a first freezing point of the extraliposomal volume below a second freezing point of the intraliposomal volume; and
cooling the composition to a predetermined temperature such that a cold slurry is formed having a plurality of ice particles within the intraliposomal volume.

19. The method of claim 18, wherein the liposomes are comprised of a lipid selected from the group consisting of 1,2-distearoyl-sn-glycero-3-phosphocholine (DSPC), egg sphingomyelin (DPSM), dipalmitoylphosphatidyl (DPPC), dicethylphosphate (DCP), L-α-phosphatidylcholine (PC), phosphatidylethanolamine, (PE), phosphatidylserine (PS), phosphatidylglycerol (PG), and a combination thereof.

20. The method of claim 19, wherein the lipid is L-α-phosphatidylcholine (PC).

21. The method of any one of claims 18-20, wherein the excipient is selected from the group consisting of a salt, an ion, Lactated Ringer's solution, a sugar, a biocompatible surfactant, a polyol, and a combination thereof.

22. The method of any one of claims 18-21, wherein the excipient is a polyol.

23. The method of claim 22, wherein the polyol is polyethylene glycol 1000 (PEG 1000).

24. The method of any one of claims 18-23, wherein the aqueous medium is comprised of water, saline, or phosphate-buffered saline (PBS).

25. The method of any one of claims 18-24, wherein the intraliposomal volume is between about 20% and 50% of a total volume of the composition.

26. The method of claim 25, wherein the intraliposomal volume is about 38% of the total volume of the composition.

27. The method of claim 25, wherein the intraliposomal volume is about 43% of the total volume of the composition.

28. The method of any one of claims 18-27, wherein the first freezing point of the extraliposomal volume is between about −20° C. and about −10° C.

29. The method of any one of claims 18-28, wherein the second freezing point of the intraliposomal volume between about −2° C. and about 0° C.

30. The method of any one of claims 18-29, wherein an average freezing point of a total volume of the composition comprising the first volume, the second volume, and the liposomes, is between about −10° C. and about −5° C.

31. The method of any one of claims 18-30, wherein the ice particles comprise between about 30% by weight and about 50% by weight of the biomaterial.

32. The method of any one of claims 18-31, wherein the predetermined temperature is between about −20° C. and −5° C.

33. The method of any one of claims 18-32, wherein a bilayer configuration of the liposomes is chosen from the group consisting of unilamellar vesicles, multilamellar vesicles, oligolamellar vesicles, multivesicular vesicles, and a combination thereof.

34. The method of any one of claims 18-33, wherein the liposomes have an average diameter of between about 0.1 μm and about 2 μm.

35. A method of making a flowable and injectable encapsulated ice solution, the method comprising:
providing a plurality of biodegradable liposomes configured to form vesicles selected from the group consisting of multilamellar, oligolamellar, multivesicular, giant unilamellar, large unilamellar, small unilamellar, or a combination thereof;
entrapping water within at least two of the plurality of liposomes to generate liposomes filled with a first volume comprising water;
adding an excipient to an extraliposomal second volume separated from the first volume, wherein the excipient alters the freezing point of the second volume relative to the first volume;
freezing the plurality of filled liposomes to generate a plurality of ice particles inside the filled liposomes; and
controlling an average diameter of each of the plurality of ice particles to a predetermined size.

36. The method of claim 35, wherein the first volume is between about 20% and 50% of a total volume of the composition.

37. The method of claim 36, wherein the first volume is about 38% of the total volume of the composition.

38. The method of claim 36, wherein the first volume is about 43% of the total volume of the composition.

39. The method of any one of claims 35-38, wherein the excipient is PEG 1000.
